(19) United States
(12) Patent Application Publication
(10) Pub. No.: US 2005/0211928 A1
(43) Pub. Date: Sep. 29, 2005

(54) Charged-particle-beam lithographic system

(75) Inventor: Yasuhiro Kumano, Tokyo (JP)

Correspondence Address: THE WEBB LAW FIRM, P.C., 700 KOPPERS BUILDING, 436 SEVENTH AVENUE, PITTSBURGH, PA, 15219, US

(73) Assignee: JEOL Ltd., Akishima (JP)

(21) Appl. No.: 11/087,386

(22) Filed: Mar. 23, 2005

(30) Foreign Application Priority Data
Mar. 23, 2004 (JP) 2004-83762

(51) Int. Cl. A61N 5/00; G21G 5/00

(52) U.S. Cl. 250/492.22; 250/492.1

(57) ABSTRACT

A charged-particle-beam lithographic system capable of sending data to a data converter in a short time. The system has a library memory for saving a library of graphics data, a writing memory for storing data about the number of reads for reading graphics data from the library memory in succession and other graphics data, a reading processor, and the data converter. The reading processor reads out graphics data saved in the library memory according to data about the number of reads from the writing memory. The converter converts the graphics data in the library and other graphics data into data adapted for the lithographic system. A temporal storage having a higher read speed than that of the library memory is placed between the writing memory and the reading processor.

### BACKGROUND OF THE INVENTION

1. Field of the Invention

The present invention relates to a charged-particle-beam lithographic system equipped with a memory for storing a library of graphics data.

2. Description of Related Art

A charged-particle-beam lithographic system, such as an electron-beam lithographic system, is an apparatus capable of writing a desired integrated circuit (IC) pattern at a desired location on a material by electron beam irradiation. The system can fabricate quite high-density semiconductor devices.

FIG. 1 schematically shows one example of the electron-beam lithographic system. The system has an electron gun 1, a blanker 2, a blanker slit 3, and an irradiation lens 4. A first shaping slit 5 has a polygonal (e.g., square or rectangular) hole in its center. Similarly, a second shaping slit 6 has a polygonal hole in its center. A further shaping lens 7 focuses an image of the hole in the first shaping slit 5 onto the second shaping slit 6. A shaping deflector 8 deflects the electron beam passed through the first shaping slit to determine the focal position of the image of the hole in the first slit on the second shaping slit. These shaping slits 5, 6, shaping lens 7, and shaping deflector 8 together form a mechanism for varying the cross section of the electron beam. A focusing lens 9 focuses the electron beam passed through the second shaping slit 6 onto a material 10 to be written. A positioning deflector 11 determines the position of the focused beam on the material. The material is placed on a sample stage 12.

Furthermore, the system has a data memory 13 in which data about a pattern to be delineated on the material is stored. In addition, the system has a control unit 14.

A data transfer circuit 15 divides the written pattern data from the data memory 13 into data sets each corresponding to the beam spot size in accordance with an instruction from the control unit 14. The transfer circuit 15 generates data about the beam spot size and data about the irradiation position. The data about the spot size is supplied to the shaping deflector 8 via a D/A converter 16 and an amplifier 17. The data about the irradiation position is supplied to the positioning deflector 11 via a D/A converter 18 and an amplifier 19. At the same time, data about the irradiation time is sent to the blanker 2 via an irradiation time control circuit 20 and a blanking signal generation circuit 21.

A stage driver circuit 22 generates a signal to controllably drive the sample stage through a stage drive mechanism 23 according to an instruction from the control unit 14.

In the system of the construction described so far, the data transfer circuit 15 divides the written pattern data from the data memory 13 into data sets each corresponding to the beam spot size in accordance with an instruction from the control unit 14. The data transfer circuit 15 generates data about the beam spot size and data about the irradiation position. The data about the spot size is supplied to the shaping deflector 8 via the D/A converter 16 and amplifier 17. The data about the irradiation position is supplied to the positioning deflector 11 via the D/A converter 18 and amplifier 19. At the same time, data about the irradiation time is sent to the blanker 2 via the irradiation time control circuit 20 and blanking signal generation circuit 21. This sequence of operations is performed for pattern data about one field. Thus, the electron beam of desired cross-sectional shape and size is shot against successive fields on the material 10. In this way, a desired pattern is delineated.

When one field of pattern is completely delineated in this manner, an instruction from the control unit 14 is sent to the stage driver circuit 22, which in turn causes the stage drive mechanism 23 to move the sample stage 12 such that the center of the field to be delineated next is brought on the optical axis of the electron beam. Then, a pattern is delineated within the field by the electron beam in the manner described above.

The IC pattern delineated on the material is designed by CAD, for example. The pattern is once stored as CAD data on a magnetic disk 24. The CAD data stored on the disk 24 is read out by a control unit 25 and saved in a writing memory 26. At the same time, graphical data (referred to also as library graphics data) of the CAD data which is repeatedly used is saved in a library memory 27.

Referring to FIG. 2, it is assumed that data about a figure A, data about a command for reading out a library figure D twice, data about a figure B, data about a command for reading out a library figure E three times, data about a figure C, and other data are stored in the order of lithography process steps in the writing memory 26. Referring to figure 3, it is assumed that data about the figure D and data about the figures E are stored as library data.

Data sets stored in the writing memory 26 are successively sent to a reading processor 28. Accordingly, the reading processor 28 first produces the data about the figure A to a data converter 29. Then, the reading processor 28 reads the data about the library figure D from the library memory 27 using the command data for reading out the data about the library figure D twice and sends the data to the data converter 29. Subsequently, the processor reads the data about the library figure D from the library memory 27 and sends the data to the data converter 29. Then, the reading processor 28 outputs the data about the figure B to the data converter 29. Then, the processor reads the data about the library figure E from the library memory 27 using the command data for reading out the data about the library figure E three times and sends the data to the data converter 29. Subsequently, the processor reads the data about the library figure E from the library memory 27 and sends the data to the data converter 29. Subsequently, the processor reads the data about the library figure E from the library memory 27 and sends the data to the data converter 29. Then, the reading processor 28 sends the data about the figure C to the data converter 29. Graphics data sent in to the data converter 29 in this way are converted into a format adapted for lithography and sent to the control unit 14. The control unit 14 divides the graphics data sent in and stores data sets about successive fields into the data memory 13 in turn.

See, for example, U.S. Pat. No. 4,291,231

The time sequence in which the reading processor 28 outputs graphics data to the data converter 29 is illustrated in FIG. 4. That is, many time intervals take place in succession as follows: time interval Ta during which the data about the figure A is output to the data converter 29, time interval Tl during which the data about the library figure D is read from the library memory 27, time interval Td during which the data about the figure D is output to the data converter 29, time interval Tl during which the data about the library figure D is read from the library memory 27, time interval Td during which the data about the figure B is output to the data converter 29, time interval Tb during which the data about the figure D is output to the data converter 29, time interval Tl′ during which the data about the library figure E is read from the library memory 27, time interval Te during which the data about the figure E is output to the data converter 29, time interval Tl′ during which the data about the library figure E is read from the library memory 27, time interval Te during which the data about the figure E is output to the data converter 29, time interval Tl′ during which the data about the library figure E is read from the library memory 27, time interval Te during which the data about the figure E is output to the data converter 29, and time interval Tc during which the data about the figure C is output to the data converter 29.

Where an IC pattern is delineated on one material by the lithographic system, it is unlikely that the number of delineated figures is restricted to a limited number as described above (i.e., eight figures consisting of A, D, D, B, E, E, E, and C). Normally, tens of thousands of figures are used. A DRAM (dynamic random access memory) which has a slow read speed but a high recording density is used as the library memory 27, in order to achieve a large capacity. Therefore, it takes a quite long time for the reading processor 28 to output graphics data to the data converter 29, thus impeding improvement of the throughput in IC pattern delineation.

### SUMMARY OF THE INVENTION

The present invention is intended to provide a novel charged-particle-beam lithographic system free of the foregoing problems.

One charged-particle-beam lithographic system according to the present invention has: a library memory for saving data about library graphics; a writing memory for storing data about the number of reads for reading data about library graphics in succession, the writing memory also storing graphics data other than the library graphics; reading processor for reading out the data about the library graphics saved in the library memory according to data about the number of reads from the writing memory; conversion means for converting the read data about the library graphics and graphics data other than the library graphics into data adapted for the lithographic system; and a lithographic system body for delineating a pattern on a material by a charged-particle beam according to data derived from the conversion means.

This lithographic system is characterized in that a high-speed memory having a higher read speed than that of the library memory is placed between the writing memory and the reading processor.

Another charged-particle-beam lithographic system according to the present invention has: a library memory for saving data about library graphics; a writing memory for storing data about the number of reads for reading out library graphics in succession, the writing memory also storing graphics data other than the library graphics; reading processing means for reading out the data about the library graphics saved in the library memory according to data about the number of reads from the writing memory; conversion means for converting the read data about the library graphics and graphics data other than the library graphics into data adapted for the lithographic system; and a lithographic system body for delineating a pattern on a material by a charged-particle beam according to data derived from the conversion means.

This lithographic system is characterized in that a high-speed memory having a higher read speed than that of the library memory is provided to store library graphics data read from the library memory and that in a case where the same graphics data is used in succession, the reading means repeatedly reads graphics data from the high-speed memory and sends the data to the conversion means.

According to the charged-particle-beam lithographic systems of the present invention, the throughput is improved greatly.

Other objects and features of the present invention will appear in the course of the description thereof, which follows.

### BRIEF DESCRIPTION OF THE DRAWINGS

FIG. 1 is a schematic diagram of one electron-beam lithographic system;

FIG. 2 is a diagram illustrating different kinds of data and the order in which the kinds of data are stored in a writing memory;

FIG. 3 is a diagram illustrating the kinds of library data stored in a library memory;

FIG. 4 is a diagram illustrating a time sequence in which a reading processor included in the prior art system outputs graphics data to a data converter;

FIG. 5 is a schematic diagram of an electron-beam lithographic system that is one example of charged-particle-beam system of the present invention; and

FIG. 6 is a diagram illustrating a time sequence in which a reading processor included in a system according to the present invention outputs graphics data to a data converter.

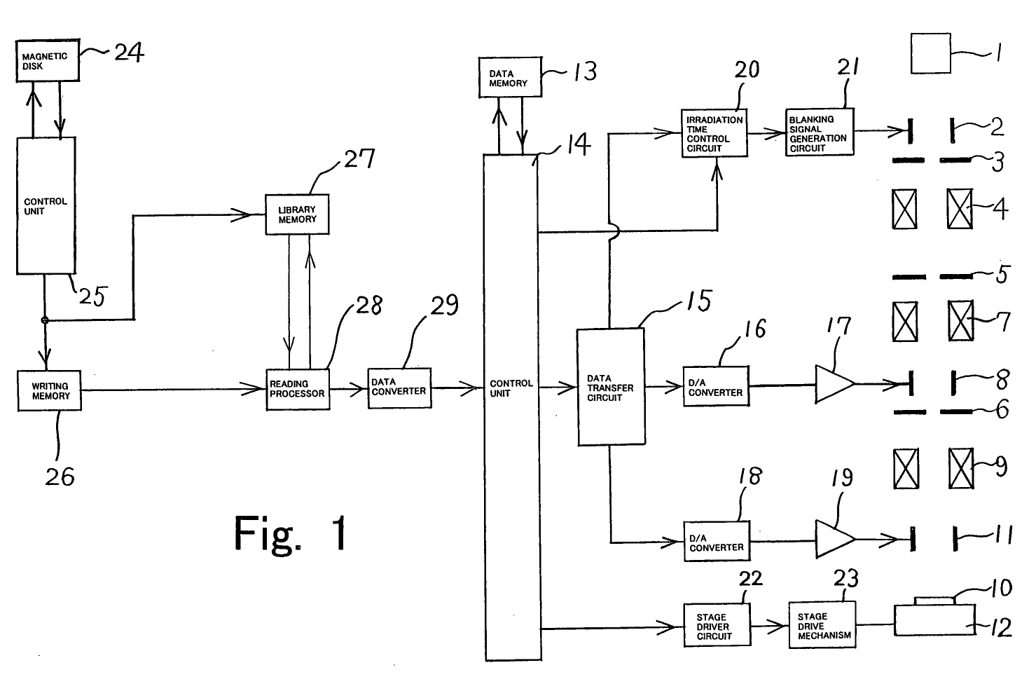

### DESCRIPTION OF THE PREFERRED EMBODIMENTS

The preferred embodiments of the invention are hereinafter described in detail with reference to the drawings.

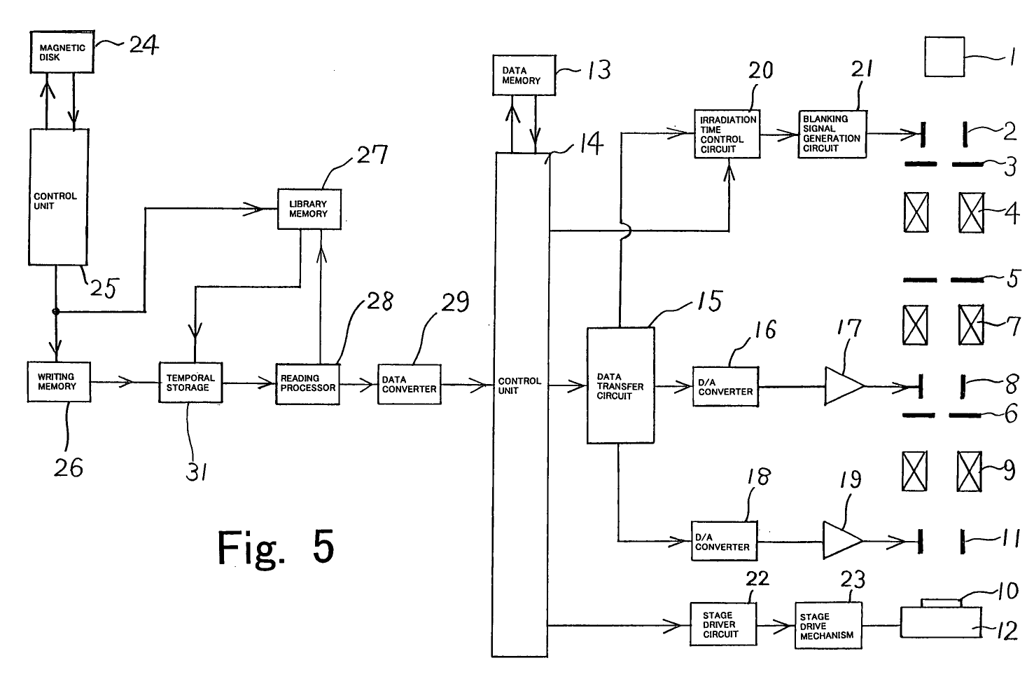

FIG. 5 schematically shows an electron-beam lithographic system that is one example of charged-particle-beam instrument of the present invention. It is to be noted that like components are indicated by like reference numerals in various figures.

In FIG. 5, indicated by reference numeral 31 is a temporal storage (also known as a working storage) consisting of a SRAM (static random access memory) that is lower in recording density but much higher in read speed than a library memory 27 having a quite high recording density. The temporal (temporary) storage 31 is connected between the output side of a writing memory 26 and the input side of the reading processor 28.

In FIG. 5, the reading processor 28 only acts to read library graphics data from the library memory 27. The graphics data read from the library memory 27 is temporarily stored in the temporal storage 31.

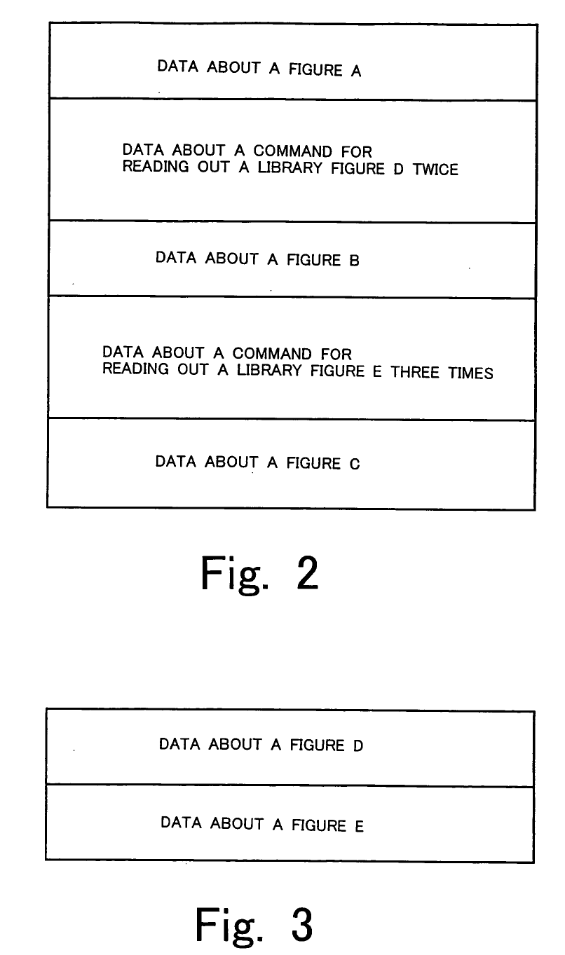

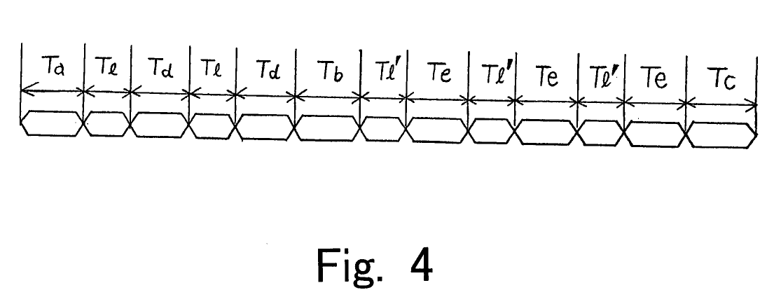

It is now assumed in the same way as the foregoing that data about figure A, data about a command for reading out library figure D twice, data about figure B, data about a command for reading out library figure E three times, data about figure C, and other data are stored in the order of lithography process steps in a writing memory 26 as shown in FIG. 2, and that data about figure D and data about figure E are saved as library data in the library memory 27 as shown in FIG. 3. Operations for sending data to the data converter 29 are described below.

Data saved in the writing memory 26 are passed through the temporal storage 31 and sent in to the reading processor 28. The reading processor 28 first outputs the data about the figure A to the data converter 29.

Then, the reading processor 28 sends a signal for reading out the data about the library figure D to the library memory 27, using the command data for reading out the data about the library figure D twice. The data about the library figure D that is read from the library memory 27 is stored in the temporal storage 31. At the same time, the data about the library figure D is output to the data converter 29. Subsequently, the reading processor 28 sends a signal for reading out the data about the figure D in the temporal storage 31 to this storage 31 rather than to the library memory 27, according to the command data for reading out data twice. Then, the data about the library figure D is read from the temporal storage 31 and output to the data converter 29.

Thereafter, the reading processor 28 sends the data about the figure B to the data converter 29. The reading processor 28 sends a signal for reading out the data about the library figure E to the library memory 27, using the command data for reading out the data about the library figure E three times. Then, the data about the library figure E read from the library memory 27 is stored in the temporal storage 31. Concurrently, the data about the library figure E is output to the converter 29. Subsequently, the reading processor 28 sends a signal for reading out the data about the figure E inside the temporal storage 31 to this storage 31. The data about the library figure E is read from the temporal storage 31 and output to the data converter 29. Then, the reading processor 28 sends a signal for reading out the data about the figure E inside the temporal storage 31 to this storage 31. The data about the library figure E is read from the temporal storage 31 and output to the data converter 29.

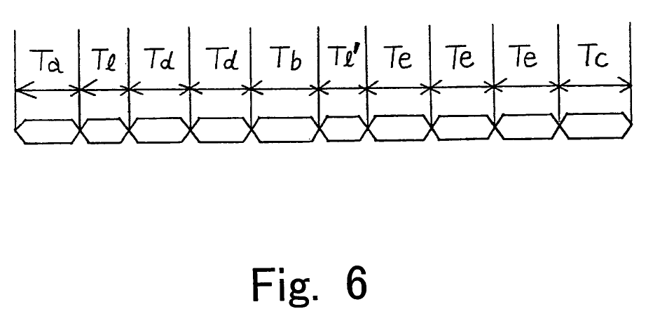

Then, the reading processor 28 sends the data about the figure C to the data converter 29. The time sequence in which graphics data is output to the data converter 29 from the reading processor 28 is illustrated in FIG. 6. That is, many time intervals take place in succession as follows: time interval Ta during which data about figure A is output to the data converter 29, time interval Tl during which data about library figure D is read from the library memory 27, time interval Td during which data about figure D is output to the data converter 29, time interval Td during which data about library figure D is output to the data converter 29, time interval Tb during which data about figure B is output to the data converter 29, time interval Tl′ during which data about library figure E is read from the library memory 27, time interval Te during which data about figure E is output to the data converter 29, time interval Te during which the data about figure E is output to the data converter 29, time interval Te during which data about the figure E is output to the data converter 29, and time interval Tc during which the data about figure B is output to the data converter 29.

Since the temporal storage 31 is made of a SRAM having a quite short read time, the time in which data about the library figures D and E is read out can be neglected compared with other times.

Where plural library graphics data sets are sent to the data converter 29 in succession in this way, it is not necessary to read out the library graphics data stored in the library memory 27 plural times in succession. Only one read suffices. Then, the data is read at high speed from the high-speed temporal storage 31. Therefore, the time taken to output graphics data to the data converter 29 is shortened greatly. As a result, the throughput in IC pattern delineation is improved greatly.

In the above embodiment, a SRAM is used as the temporal storage 31. Other kinds of memory may also be used if it has a higher read speed than the library memory 27. In addition, in the above embodiment, an electron-beam lithographic system of the variable area type is taken as an example. The present invention can also be applied to other types of electron-beam lithographic system, such as a spot electron-beam lithographic system or ion-beam lithographic system.

Having thus described my invention in the detail and particularity required by the Patent Laws, what is desired protected by Letters Patent is set forth in the following claims.

1. A charged-particle-beam lithographic system comprising:
a library memory for saving graphics data forming a library;
a writing memory for storing data about the number of reads for reading out graphics data from the library in succession, the writing memory also storing graphics data other than the graphics data in the library;
reading processing means for reading out graphics data saved in the library memory according to data about the number of reads from the writing memory;
conversion means for converting the read data about the graphics data and other graphics data into data adapted for the lithographic system;
a lithographic system body for delineating a pattern on a material by a charged-particle beam according to data derived from the conversion means; and
a high-speed memory having a higher read speed than that of the library memory placed between the writing memory and the reading processing means.

2. A charged-particle-beam lithographic system as set forth in claim 1, wherein said high-speed memory is a SRAM (static random access memory).

3. A charged-particle-beam lithographic system as set forth in claim 1, wherein said high-speed memory is a temporal storage.

4. A charged-particle-beam lithographic system comprising:
a library memory for saving graphics data forming a library;
a writing memory for storing data about the number of reads for reading out graphics data from the library in succession, the writing memory also storing graphics data other than the graphics data in the library;
reading processing means for reading out graphics data saved in the library memory according to data about the number of reads from the writing memory;
conversion means for converting the read data about the graphics data and other graphics data into data adapted for the lithographic system;
a lithographic system body for delineating a pattern on a material by a charged-particle beam according to data derived from the conversion means; and
a high-speed memory having a higher read speed than that of the library memory provided to store graphics data read from the library memory such that when the same graphics data is used in succession, said reading processing means repeatedly reads graphics data from said high-speed memory and sends the data to said conversion means.

5. A charged-particle-beam lithographic system as set forth in claim 4, wherein said high-speed memory is a SRAM (static random access memory).

6. A charged-particle-beam lithographic system as set forth in claim 4, wherein said high-speed memory is a temporal storage.
